(19) United States
(12) Patent Application Publication
(10) Pub. No.: US 2025/0222052 A1
(43) Pub. Date: Jul. 10, 2025

(54) HIGH YIELD EXTRACTION METHOD FOR AND PRODUCTS OF AMANITA MUSCARIA

(71) Applicant: PLANT SYNERGY INC., Yorklyn, DE (US)

(72) Inventor: Armand Bytton, Pasadena, CA (US)

(21) Appl. No.: 18/409,123

(22) Filed: Jan. 10, 2024

(51) Int. Cl. A61K 36/07; A24B 15/167; A24D 1/02; A24D 3/14; A24D 3/17; A61K 31/00

(52) U.S. Cl. CPC A61K 36/07; A24B 15/167; A24D 1/022; A24D 3/14; A24D 3/17; A61K 31/658; A61K 2236/17; A61K 2236/33; A61K 2236/331; A61K 2236/35; A61K 2236/51; A61K 2236/53

(57) ABSTRACT

A method for extracting an Amanita muscaria mushroom includes the steps of harvesting and drying the Amanita muscaria mushroom, extracting a whole raw extract from the Amanita muscaria mushroom in a solvent, filtering the solvent-Amanita muscaria mushroom mixture to separate the spent Amanita muscaria mushroom matter and the solvent from the whole raw extract, separating a first portion of the whole raw extract from a second portion of the whole raw extract, purifying the first portion of the whole raw extract to concentrate target molecules therein, wherein the target molecules comprise muscimol and ibotenic acid, and mixing the first portion back into the second portion to produce a mixture.

### FIELD OF THE INVENTION

The present invention relates to a method for high yield extraction of fungal biomass. More particularly, the present invention relates to a method for high yield extraction of Amanita muscaria fungal species and products made therefrom for medicinal and commercial use.

### BACKGROUND

Phytochemicals are defined as chemical compounds derived from or naturally produced generally by plants, whereas mycochemicals are defined as chemical compounds derived from or naturally produced by a fungus. Fungi are organisms that include reproductive organs commonly known as mushrooms. Amanita muscaria is a species of fungus that produces mushrooms with red or yellow caps spotted with white dots. Amanita muscaria grows at the base of pine and birch trees in temperate regions. Amanita muscaria contains the mycochemicals ibotenic acid and muscimol, among a large number of others, and has known hallucinogenic properties.

Crude plant or fungal extracts or plant or fungal material traditionally consumed by ingestion contain many compounds, phytochemicals in plants, and mycochemicals in fungi, such as secondary metabolites, which may affect many targets in the body simultaneously. These complex mixtures of phytochemicals or mycochemicals may have numerous, synergistic effects in the body and “minor” metabolites may likely affect targets that have an equilibrizing effect that can, for example, mitigate side effects as compared to a synthetic pure pharmaceutical. The overall effect, generally referred to as the “entourage effect,” may be more desirable and more potent than that of only one compound alone. For example, Amanita muscaria produces a large number of mycochemicals including, for example without limitation, muscarine, bufotenin, muscimol, ibotenic acid, and muscazone, among many others.

In another example, the cannabis plant produces hundreds of phytochemicals with their own therapeutic potential and the capability to induce synergic responses when combined due to the “entourage effect.” Often the entourage effect of the supportive but undescribed molecules in a whole plant extract, whether from cannabis or Amanita muscaria or any plant, fungus, or natural product, may potentiate particular target molecules, yielding a more desirable result. For instance, one study tested the anti-tumor efficacy of the botanical drug preparation (whole plant extract) versus pure tetrahydrocannabinol (THC) against breast cancer.

https://www.sciencedirect.com/science/article/abs/pii/S0006295218302387. The results provide that while pure THC acted by activating cannabinoid CB2 receptors and generating reactive oxygen species, the whole plant extract modulated different targets and mechanisms of action. These, and other similar experimental results suggest that standardized cannabis drug preparations, rather than pure cannabinoids, could be considered as part of the therapeutic armamentarium to manage various diseases and in this particular study, breast cancer. However, processes for extraction of the phytochemicals and mycochemicals related to how they influence the entourage effect are under advanced study and not yet available for the scientific and global community.

Dried Amanita muscaria can be acquired from international and/or local sources, and can contain varying concentrations of target molecules from batch to batch depending on quality of harvest. Upon analysis after harvesting, Amanita muscaria may contain a wide range of active molecules, highly dependent on the time of year and/or climate conditions for a particular year. Some of the target molecules of interest are the mycochemicals, including, for example without limitation, muscarine, bufotenin, muscimol, ibotenic acid, and muscazone.

Muscimol, a mycochemical and one of the target molecules of interest present in Amanita muscaria, is on average only a small percentage of the total dry weight of the fruiting body, while ibotenic acid, another mycochemical, is generally much more abundant. Preparation of an extract utilizing a variety of decarboxylation methods converts ibotenic acid into muscimol. The chemical conversion of ibotenic acid to muscimol is illustrated in FIG. 1.

Hybridization of Fungi species, such as those in the Amanita genus, may occur naturally. Hybridized medicinal or non-medicinal natural products from plants (or fungi) provide many benefits, including phytochemical (or mycochemical) and phytonutrient (or myconutrient) enriched properties and increased yields of certain phytochemicals (or mycochemicals) that have yet to be further researched and discovered. Such plants or fungi may be desirable in embodiments of the present extraction method for the unique outputs and thus compositions achievable.

Two receptors, CB1 and CB2, of the endocannabinoid system have been identified so far, with consensus being that there very well could be more yet to be discovered. CB1 and CB2 receptors are both G-protein coupled receptors. Endocannabinoids act as retrograde signaling messengers that stimulate presynaptic CB1 receptors on neurons in the brain. This stimulation results in regulation of ion channel activities, inhibition of adenylate cyclase activity, and activation of the mitogen-activated protein kinase cascade.

CB1 receptors are the most abundant receptors in the mammalian brain, and also have been detected in other peripheral tissues and cells such as testis, eye, urinary bladder, ileum and adipocytes, though in lower concentrations than in the brain. CB1 receptors are found in brain areas related to the control of anxiety and emotional perception. In rats, CB1 receptors have been found with 5-HT transporters in the amygdale, suggesting that they mediate 5-HT release therefore possibly a target for the treatment of disorders related to mood, anxiety and cognition.

Despite the vast forward progress in natural products medicinal research, the properties (specific naturally occurring chemical compounds) and effects of compositions made from extracts of the fungal species of Amanita muscaria and current extraction processes to advance these properties and effects regarding how they affect the biochemical reactions of the endocannabinoid system remain in need of further development.

### BRIEF SUMMARY OF THE INVENTION

In a first aspect of the invention, a method for extracting an Amanita muscaria mushroom comprises the steps of harvesting and drying the Amanita muscaria mushroom, extracting a whole raw extract from the Amanita muscaria mushroom in a solvent, filtering the solvent-Amanita muscaria mushroom mixture to separate the spent Amanita muscaria mushroom matter and the solvent from the whole raw extract, separating a first portion of the whole raw extract from a second portion of the whole raw extract, purifying the first portion of the whole raw extract to concentrate target molecules therein, wherein the target molecules comprise muscimol and ibotenic acid, and mixing the first portion back into the second portion to produce a mixture.

In a second aspect of the invention, a composition comprises a whole raw extract extracted from an Amanita muscaria mushroom, and a purified whole raw extract extracted from an Amanita muscaria mushroom, wherein the purified whole raw extract comprises a concentration of target molecules that is at least double the concentration found in the whole raw extract, wherein the target molecules comprise muscimol and ibotenic acid.

Numerous other features and advantages of the present invention will be readily apparent to those of ordinary skill in the art upon reading of the following detailed description of presently preferred, but nonetheless illustrative, embodiments of the present invention when taken in conjunction with the accompanying figures. The invention is capable of other embodiments and of being practiced and carried out in various ways. Also, it is to be understood that the phraseology and terminology employed herein are for the purpose of description and should not be regarded as limiting. Those skilled in the art will appreciate that the invention described herein is susceptible to variations and modifications other than those specifically described. It is to be understood that the invention includes all such variations and modification which fall within its spirit and scope.

### BRIEF DESCRIPTIONS OF THE FIGURES

FIG. 1 illustrates the chemical conversion of ibotenic acid to muscimol.

FIG. 2 illustrates peaks for various target molecules, including muscimol, being separated from a whole raw extract of Amanita muscaria via flash chromatography.

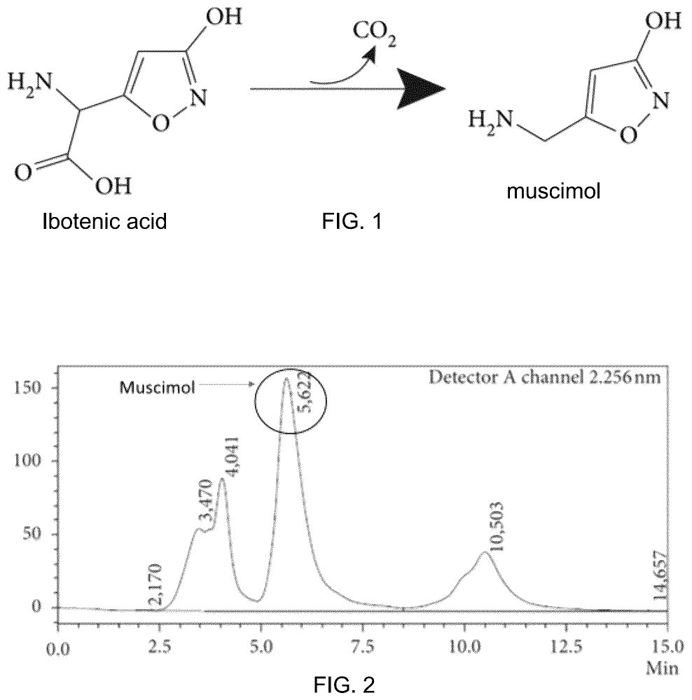

### DETAILED DESCRIPTION

Hereinafter, methods of high yield extraction of fungal matter of an Amanita muscaria mushroom and products made therefrom for medicinal and commercial use have been described.

Following the completion of the harvesting and drying of the Amanita muscaria mushroom, in an embodiment dried Amanita muscaria mushroom matter is extracted, for example by being dissolved in a solvent, for example without limitation, water, an alcohol, or a ketone (the choice of which being contingent upon the method of ‘separation’ chosen and the criteria of the equipment being used). In an embodiment the mixture of Amanita muscaria mushroom matter and solvent is prepared into a whole raw extract, for example using water or ethanol, for example by Soxhlet extraction. In an embodiment the water or ethanol is then removed, and the whole raw extract subjected to acidification (for example using H2SO4 or formic acid) and filtering. In an embodiment an organic solvent (such as hexane) is used to wash the acidic solution and the organic phase discarded, followed by adding an ammonia solution to neutralize it and result in an alkaline solution. If need be the latter can be further extracted with dichloromethane.

The whole raw extract so produced is a full spectrum extract of Amanita muscaria containing the same variety and myriad of compounds and mycochemicals as in the Amanita muscaria mushroom matter and thus also provides the “entourage effect” provided by the Amanita muscaria mushroom itself. In an embodiment the whole raw extract is collected into a flask and evaporated under vacuum to yield a liquid. In an embodiment a first portion of the whole raw extract is set aside for a later recombination step while a second portion of the whole raw extract is processed further by a series of purification steps. In an embodiment a ratio of the first and second portions of the whole raw extract is about 1:1; however, on other embodiments the ratio can be less than or greater than 1:1.

In an embodiment the second portion of the whole raw extract is purified and concentrated, for example, by chromatography, in order to isolate, concentrate and optimize desired target molecules, which are then resuspended into the first portion of the whole raw extract to yield a mixture having concentrated target molecules. In an embodiment the concentrated target molecules comprise, for example without limitation, muscimol and ibotenic acid. In an embodiment the purified second portion of the whole raw extract comprises a concentration of target molecules that is, for example without limitation, at least double the concentration found in the first portion of the whole raw extract. The mixture having concentrated target molecules represents a man-made product composition that takes advantage of the target molecules potency while utilizing the heightened “entourage effect” of the many undescribed mycochemicals found in the first portion of the whole raw extract.

In an embodiment, during the extraction phase, the bulk of mycochemicals soluble in the solvent are isolated away from bulk fibers, structural fungal material, and cell debris, which are disposed of in proper chemical waste containers. In an embodiment the remaining whole raw extract is then, for example, subjected to chromatography steps that isolate desired target molecules away from the myriad of other mycochemicals extracted by the extracting step. Target eluents are collected for further processing and the waste eluent is collected/disposed of in proper chemical waste containers.

In an embodiment the purification of the first portion of the whole raw extract is carried out by passing the solvent and the first portion of the whole raw extract over or through a process selected from the group consisting of column chromatography comprising a reverse stationary phase, a normal stationary phase or combination thereof, ion-exchange chromatography, gel-permeation (molecular sieve) chromatography, affinity chromatography, paper chromatography, thin-layer chromatography, gas chromatography, dye-ligand chromatography, hydrophobic interaction chromatography, pseudoaffinity chromatography, and high-pressure liquid chromatography (HPLC).

In an embodiment, the solvent used in the extracting step is a known food grade solvent, for example including but not limited to ethanol, methanol, hydroalcohol, acetone, acetonitrile, hexane, heptane, hexane, chloroform, dichloromethane, water, and mixtures thereof. For example, in an embodiment the solvent is ethanol. In an embodiment, the amount of solvent added is in a ratio of about 1:1 with the fungal biomass.

Solvents are chosen based on equipment specifications and the relative covalency of the compounds. There are many covalent compounds which could be used in this method. For example, ethanol, which is used in an embodiment, evaporates very easily under low vacuum, and thus, in this example the equipment cycling parameters are chosen based on the need for evaporation of the ethanol solvent. In an embodiment, the solvent is removed by a process, for example including but not limited to distillation, open-dish evaporation, reduced-pressure evaporation, rotary evaporation, vacuum, lyophilization, and/or a combination of these processes. In an embodiment the solvent is removed by rotary evaporation.

In an embodiment other mycochemicals, phytochemicals, or other natural products such as cannabidiol (CBD) may be added to the mixture of purified and unpurified whole raw extract described hereinabove. In an embodiment the CBD is added to the Amanita muscaria mushroom and the solvent during the extracting step. In another embodiment the CBD is added to the mixture produced by the above described method. In an embodiment CBD is added during both the extracting and mixing steps. The addition of CBD at either or both of the extracting and mixing steps results in a CBD enhanced mixture. In an embodiment the CBD source can be selected from, but is not limited to, hemp plants, crude hemp oil, kief, nabiximol, epidiolex, and combinations thereof. In an embodiment the cannabidiol source is crude hemp oil.

Cannabinoids can be defined as any extract, isolate, or derivative of the Cannabaceae family of plants, comprising the Cannabis genus, and the species Cannabis sativa., Cannabis indica, and Cannabis ruderalis, or any combination thereof, produced by—or as part of—a synthetic transformative process involving any other combination of cannabinoids or their derivatives. This starting material can be obtained from any commercially available sources, or synthesized in situ, at a time prior to inception of the methods described herein. In some embodiments, other herbal products, including ginger, turmeric, holy basil, and one or more cannabinoids could be added during either or both of the extracting and mixing steps instead of or in addition to CBD, such as, for example without limitation, cannabielsoin (CBE), cannabinol (CBN), cannabichromene (CBC), cannabigerol (CBG), and combinations thereof, thus leading to different ultimate combinations.

Once the target molecules in the purified second portion of the whole raw extract are purified and concentrated to an acceptable extent and added back to the first portion of the whole raw extract to produce the mixture having concentrated target molecules, the mixture may be utilized in liquid form or dried to produce a powder or a resin. The primary difference between the mixture in a powder form and the mixture in a resin form is that the powder form would have the highest concentration of mycochemicals due to the near complete removal of water as compared to a resin or liquid, but can be manipulated to result in any desired concentration of mycochemicals by the addition of water or any other hydrophilic solvent such as, for example without limitation, glycerin or propylene glycol.

Regardless of the form, either as a liquid or a powder or a resin, the mixture having concentrated target molecules provides optimized and concentrated target molecules blended with whole raw extract to take advantage of potency of the concentrated target molecules while utilizing the entourage effect of the many undescribed mycochemicals found in the whole raw extract.

For example, in an embodiment the mixture having concentrated target molecules in powder form can be “sprinkled” on other herbal products (i.e., ginger, turmeric, holy basil, among other known products) or mixed with other phytochemicals, mycochemicals, phytonutrients, myconutrients, phytomaterials, or mycomaterials by a consumer, or packed into capsules or tablets. In an embodiment the mixture having concentrated target molecules in either powder, resin, or liquid form can be used in medicinal or commercial compositions including, for example without limitation, application on or in known delivery vehicles, such as vaporizing liquids, electronic liquids (e-liquids) for vaporization, medicinal compositions, kief, saturated or infused rolling papers, filters for tobacco and vaporizing equipment, and internasal delivery systems such as a spray.

In an embodiment the mixture having concentrated target molecules in either powder, resin, or liquid form is infused into rolling papers. In an embodiment the weight of the mixture in powder or resin form being infused into each rolling paper is in a range between 10 mg and 250 mg.

In an embodiment the method further includes solubilizing the mixture having concentrated target molecules in a second solvent, such as, for instance an electronic vaporizer liquid (“e-liquid”) or in a second solvent before putting in an e-liquid. The second solvent could be a known food grade solvent, including but not limited to ethanol, methanol, hydroalcohol, acetone, acetonitrile, hexane, heptane, hexane, chloroform, dichloromethane, water, and mixtures thereof. In an embodiment the second solvent liquid may be the same or different from the extracting solvent used in the extracting step. In an embodiment of the method, the amount of second solvent liquid added is in a ratio of about 1:1 with the mixture having concentrated target molecules.

In an embodiment the process of separation and isolation of mycochemicals from Amanita muscaria species via flash chromatography (see FIG. 2) can be carried out after filtration. Referencing relevant alkaloid reference standards in order to properly identify the target molecules, in an embodiment we use a Biotage® Flash chromatography Isolera® Single Channel system or Biotage Selekt® Flash Chromatography system utilizing a C18 reverse phase silica column and detected at a UV wavelength of 254 nm. Some solvents used may be Dichloromethane, acetone, methanol, and acetonitrile. In an embodiment methanol and 1% acetic acid or formic acid in water mixed over a 10 minute gradient program, passes through the C18 column, separating out target molecules by detectable peaks on the chromatogram. Following the separation of components by flash chromatography, collected eluents are then filtered and processed again through the Flash Chromatography unit (Biotage® |solera® or Selekt® chromatography system), utilizing resins with different characteristics such as charge or hydrophobicity yielding pure target molecules of bufotenin, muscimol, ibotenic acid, and muscazone (as well as others). These concentrated molecules can then be re-suspended into the whole raw extract to produce the desired mixture at multiple units of concentration.

In an embodiment the purified second portion of the whole raw extract comprises a concentration of target molecules that is at least double the concentration found in the first portion of the whole raw extract. In an embodiment the purified second portion of the whole raw extract comprises a concentration of target molecules that is more than double, three times, four times, five times, six times, seven times, eight times, nine times, or ten or more times the concentration found in the first portion of the whole raw extract.

In some embodiments, medicinal uses for the mixture having concentrated target molecules include, but are not limited to, compositions for treatment of psychological disorders ranging from depression to anxiety. The resulting compositional output of the present method may be used to treat disorders in the form of a sleep aid, a pain reliever or a performance enhancer, or be used to treat premenstrual syndrome and eating disorders.

Various delivery vehicles can be used, as discussed above, and the choice of the same will depend on various factors such as, but not limited to, the desired result or ease of manipulation or even the speed of delivery or efficacy, and the concentration of the final composition of the present method could be dictated by the desired effect, the subject, what is being treated, a combination of any of the same and other factors.

Numerous modifications to the present invention will be apparent to those skilled in the art in view of the foregoing description. It is not desired to limit the invention to the exact method, construction, or operation shown and described, and accordingly, all suitable modifications and equivalents may be resorted to, falling within the scope of the invention. Accordingly, this description is to be construed as illustrative only of the principles of the invention and is presented for the purpose of enabling those skilled in the art to make and use the invention and to teach the best mode of carrying out same. The exclusive rights to all modifications which come within the scope of the appended claims are reserved. All patents, patent publications and applications, and other references cited herein are incorporated by reference herein in their entirety.

I claim:

1. A method comprising the steps of:
harvesting and drying an Amanita muscaria mushroom;
extracting in a solvent a whole raw extract from the Amanita muscaria mushroom;
filtering the solvent-Amanita muscaria mushroom mixture to separate a spent Amanita muscaria mushroom matter and the solvent from the whole raw extract;
separating a first portion of the whole raw extract from a second portion of the whole raw extract;
purifying the first portion of the whole raw extract to concentrate target molecules therein, wherein the target molecules comprise muscimol and ibotenic acid; and
mixing the first portion back into the second portion to produce a mixture.

2. The method according to claim 1, wherein the solvent is selected from the group consisting of ethanol, methanol, hydroalcohol, acetone, acetonitrile, hexane, heptane, hexane, chloroform, dichloromethane, water, and mixtures thereof.

3. The method according to claim 1, wherein the filtering of the solvent is by a process selected from the group consisting of distillation, open-dish evaporation, reduced-pressure evaporation, rotary evaporation, vacuum, lyophilization, and combinations thereof.

4. The method according to claim 1, wherein the step of purifying the first portion of the whole raw extract comprises passing the solvent and the first portion of the whole raw extract over or through a process selected from the group consisting of column chromatography comprising a reverse stationary phase, a normal stationary phase or combination thereof, ion-exchange chromatography, gel-permeation (molecular sieve) chromatography, affinity chromatography, paper chromatography, thin-layer chromatography, gas chromatography, dye-ligand chromatography, hydrophobic interaction chromatography, pseudoaffinity chromatography, and high-pressure liquid chromatography (HPLC).

5. A vaporizing liquid comprising the mixture produced according to the method of claim 1.

6. A medicinal composition comprising the mixture produced according to the method of claim 1.

7. Rolling papers comprising the mixture produced according to the method of claim 1.

8. A filter for tobacco or vaporizing equipment, the filter comprising the mixture produced according to the method of claim 1.

9. The method according to claim 1, wherein the method further comprises adding Cannabidiol (CBD) to the Amanita muscaria mushroom and the solvent during the extracting step, wherein the addition of CBD results in a CBD-enhanced mixture.

10. The method according to claim 9, wherein a source for the CBD is selected from the group consisting of hemp plants, crude hemp oil, kief, nabiximol, epidiolex, and combinations thereof.

11. A vaporizing liquid comprising the CBD enhanced mixture produced according to the method of claim 9.

12. A medicinal composition comprising the CBD enhanced mixture produced according to the method of claim 9.

13. Rolling papers comprising the CBD enhanced mixture produced according to the method of claim 9.

14. A filter for tobacco or vaporizing equipment, the filter comprising the CBD-enhanced mixture produced according to the method of claim 9.

15. A composition, comprising:
a whole raw extract extracted from an Amanita muscaria mushroom; and
a purified whole raw extract extracted from an Amanita muscaria mushroom, wherein the purified whole raw extract comprises a concentration of target molecules that is at least double the concentration found in the whole raw extract, wherein the target molecules comprise muscimol and ibotenic acid.

16. The composition of claim 15, wherein a ratio of the purified whole raw extract to the whole raw extract is about 1:1.

17. A vaporizing liquid comprising the composition of claim 15.

18. A medicinal composition comprising the composition of claim 15.

19. Rolling papers comprising the composition of claim 15.

20. A filter for tobacco or vaporizing equipment, the filter comprising the composition of claim 15.
